(19) United States
(12) Patent Application Publication
(10) Pub. No.: US 2019/0045831 A1
(43) Pub. Date: Feb. 14, 2019

(54) NOVEL COATING SYSTEM (II)

(71) Applicant: DSM IP ASSETS B.V., Heerlen (NL)

(72) Inventors: Elger FUNDA, Kaiseraugst (CH); Odile KRAINZ, Kaiseraugst (CH); Simone KÖNIG-GRILLO, Kaiseraugst (CH); Zdravka MISIC, Kaiseraugst (CH); Markus NOWOTNY, Kaiseraugst (CH); Loni SCHWEIKERT, Kaiseraugst (CH)

(21) Appl. No.: 16/078,293

(22) PCT Filed: Feb. 21, 2017
(86) PCT No.: PCT/EP2017/053859

§ 371 Date: Aug. 21, 2018

(30) Foreign Application Priority Data
Feb. 26, 2016 (EP) 16157687.1

(51) Int. Cl. A23P 20/15; A23P 10/30; A23L 33/12; A23L 33/18; A23D 9/05; A23K 20/147; A23K 20/158; A61K 9/50; A61K 31/202

(52) U.S. Cl. CPC A23P 20/15; A23P 10/30; A23L 33/12; A23L 33/18; A23V 2002/00; A23K 20/147; A23K 20/158; A61K 9/5052; A61K 31/202; A23D 9/05

(57) ABSTRACT

The present patent application relates to a novel coating system for coating solid particles, which comprise PUFAs (and/or salts thereof), wherein the coating comprises at least one protein hydrolysate. Furthermore it relates to compositions coated with such a coating system and the use of such compositions in the production of food, feed, dietary supplements and/or pharmaceutical products.

The present patent application relates to a novel coating system for coating solid particles, which comprise PUFAs (and/or salts thereof), wherein the coating comprises at least one protein hydrolysate. Furthermore it relates to compositions coated with such a coating system and the use of such compositions in the production of food, feed, dietary supplements and/or pharmaceutical products.

Poly-unsaturated fatty acids (PUFAs) as well as their salts (such the Na, K or Ca salts) are very well known compounds for a healthy diet (especially the omega-3 fatty acids). The PUFAs (especially the omega-3 fatty acids) have a variety of health benefits such as i.e. health benefits against cardiovascular diseases (CVDs) including well-established hypotriglyceridemic and anti-inflammatory effects.

PUFAs can be found in a variety of plants and animals. A very good source of omega-3 fatty acids are i.e. fish.

Alternatively, PUFAs can also be produced synthetically.

Due to the fact that a lot of consumers do not like fish, it is very common to add PUFAs (and/or salts thereof) to other dietary products (enrich these products with PUFA).

The problem with the PUFAs as well as with their salts is, that they have strong tendency to oxidise. This results in a loss of the PUFAs in the product and secondly (even worse) in the development of a strong and very unpleasant smell.

With an increasing number of double bonds, the PUFAs are subject to increasing oxidative degradation and development of undesirable “off-flavors”, mainly fishy and rancid smell and taste.

Volatile degradation products cause off-flavor even at very low concentration. Sensory properties of a product may become unacceptable even before a loss of PUFAs can be analytically detected.

Stabilization of PUFAs by formation of inclusion complexes is a well-known technique for encapsulation of hydrophobic substances. However, as delivery form for PUFAs these inclusion complexes have some serious drawbacks.

Due to the fact, that inclusion complexes are formed in a molar 1:1 ratio. This limits the maximum payload of the obtained powder.

Finally, the main disadvantage of encapsulated PUFA forms isn't the loss of PUFA oxidation but formation of very small amounts of volatile degradation products, leading to fishy, painty or rancid smell of the product. As Hadaruga et al (Hadaruga, Daniel I.; Unlusayin, Mustafa; Gruia, Alexandra T.; Birau, Cristina; Rusu, Gerlinde; Hadaruga, Nicoleta G., Beilstein Journal of Organic Chemistry (2016), 12, 179-191, doi: 10.3762/bjoc.12.20) have shown, inclusion complexes can reduce but not completely prevent PUFA oxidation and therefore the problem of the small amounts of volatile degradation products isn't solved by encapsulating PUFA in inclusion complexes.

Now the goal of the present invention was to provide a powderous formulation with high content of PUFA (usually more than 10 weight-% (wt-%), based on the total weight of the powderous formulation), which has significantly improved stability in regard to the development of the undesirable “off-flavors.

In other words, the formulation having a high content of PUFA (and/or salts thereof) according to the present invention does not smell (fishy) even after storage.

Surprisingly it was found that when a coating system, which comprises at least 10 wt-% of at least one protein hydrolysate, based on the total weight of the coating system, is used, the so coated solid particles comprising PUFA (and/or salts thereof) have no (or a significantly reduced) tendency of developing undesirable “off-flavors”

Therefore the present invention relates to a coating system (CS) comprising at least 10 wt-% of at least one protein hydrolysate, based on the total weight of the coating system.

Protein hydrolysates are prepared by splitting a protein with acid, alkali, or enzyme. The protein can be from any source. It can be a plant source or an animal source. The protein can also be modified before it is hydrolysated.

Therefore the present invention relates to a coating system (CS1) which is coating system (CS), wherein the protein hydrolysates are prepared by splitting a protein with acid, alkali, or enzyme.

Therefore the present invention relates to a coating system (CS2) which is coating system (CS) or (CS1), wherein the protein (of the protein hydrolysate) is sourced from a plant or from an animal.

Therefore the present invention relates to a coating system (CS3) which is coating system (CS), (CS1) or (CS2), wherein the protein (of the protein hydrolysate) is modified before it is hydrolysated.

The coating system can comprise further auxiliary compounds, such as binders, forming compounds (such as hydrocolloids, which can be either a polysaccharide or a protein), plasticizers (such as sugars like sucrose or a sugar derivative (mannitol, sorbitol), glycerol, mono- and diglyceride, acetylated monoglyceride, polyethylene glycol (PEG), polypropylene glycol), fillers, dyestuffs, flavors, antioxidants, etc.

These ingredients are used in an amount of up to 90 wt-%, based on the total weight of the coating system.

Therefore the present invention relates to a coating system (CS4) which is coating system (CS), (CS1), (CS2) or (CS3), comprising at least one auxiliary compound (up to 90 wt-%, based on the total weight of the coating system, of at least one auxiliary compound).

Therefore the present invention relates to a coating system (CS4′) which is coating system (CS4) wherein the at least one auxiliary compound is chosen from the group consisting of binders, forming compounds (such as hydrocolloids, which can be either a polysaccharide or a protein), plasticizers (such as sugars like sucrose or a sugar derivative (mannitol, sorbitol), glycerol, mono- and diglyceride, acetylated monoglyceride, polyethylene glycol (PEG), polypropylene glycol), fillers, dyestuffs, flavors and antioxidants.

Preferred plasticizers according to the present invention are sugars like sucrose or a sugar derivative (mannitol, sorbitol), glycerol, mono- and diglyceride, acetylated monoglyceride, polyethylene glycol (PEG), polypropylene glycol. Preferably the PEG has a molecular weight between 200 and 6000. It is clear that one single plasticizer can be used as well as mixtures of two and more plasticizers.

Therefore the present invention relates to a coating system (CS4), which is coating system (CS4) or (CS4′), comprising at least plasticizer chosen from the group consisting of sucrose or a sugar derivative (mannitol, sorbitol), glycerol, mono- and diglyceride, acetylated monoglyceride, polyethylene glycol (PEG), polypropylene glycol (preferably the PEG has a molecular weight between 200 and 6000).

The coating system according to the present invention is used for coating a (particulate) solid formulation comprising PUFAs (and/or salts thereof). Such a coated system comprises a core (comprising the PUFA or a mixture of various PUFAs) and the coating system.

Therefore the present invention also relates to a coated composition (CC) comprising
(a) a core, which is a sold formulation comprises at least one PUFA (and/or salts thereof) and
(b) a coating system, comprising
   (i) at least one protein hydrolysate.

PUFAs are classified according to the position of the double bonds in the carbon chain of the molecule as n-9, n-6 or n-3 PUFAs. Examples of n-6 PUFAs are linoleic acid (C18:2), arachidonic acid (C20:4), γ-linolenic acid (GLA, C18:13) and di-homo-γ-linolenic acid (DGLA, C20:3). Examples of n-3 PUFAs are α-linolenic acid (C18:13), eicosapentaenoic acid (EPA, C20:5), and docosahexaenoic acid (DHA, C22:6). Especially EPA and DHA have attracted interest of the food industry in recent years. The most available sources of these two fatty acids are fish and the marine oils extracted from them. Suitable PUFA salts are the sodium, potassium and/or calcium salts.

Therefore the present invention also relates to a coated composition (CC1), which is coated composition (CC), wherein the at least one PUFA is chosen from the group consisting of n-9, n-6 or n-3 PUFAs and/or the salts thereof (especially the sodium, potassium or calcium salts).

Therefore the present invention also relates to a coated composition (CC1′), which is coated composition (CC) of (CC1), wherein the at least one PUFA is chosen from the group consisting of linoleic acid (C18:2), arachidonic acid (C20:4), γ-linolenic acid (GLA, C18:13), dihomo-γ-linolenic acid (DGLA, C20:3), α-linolenic acid (C18:13), eicosapentaenoic acid (EPA, C20:5) and docosahexaenoic acid (DHA, C22:6) and/or the salts thereof (especially the sodium, potassium or calcium salts).

All the preferences for the coating system apply to the above mentioned compositions.

In addition the core can comprise further ingredients, usually additives, which are used in the production of such compounds or additives which are useful for products in which the compositions according to the present invention are incorporated. The core of the composition can be in any form. It can for example be in the form of beadlet comprising the active ingredient. A suitable beadlet, which can be coated by the coating system according to the present invention can be found in WO 2007/045488.

Furthermore the present invention relates to a coated composition (CC2), which is coated composition (CC), (CC1) or (CC1′) comprising
(i) 70 to 99.5 wt-%, based on the total weight of the composition, of core and
(ii) 0.5 to 30 wt-%, based on the total weight of the composition, of coating system.

The core usually contains at least 10 wt-% of PUFA (and/or salts thereof), based on the total weight of the core; preferably at least 15 wt-% up to 50 wt-%.

Therefore the present invention relates to a coated composition (CC3), which is coated composition (CC), (CC1), (CC1′) or (CC2), wherein the core comprises at least 10 wt-% of PUFA (and/or salts thereof), based on the total weight of the core.

Therefore the present invention relates to a coated composition (CC3′), which is coated composition (CC), (CC1), (CC1′) or (CC2), wherein the core comprises 15 wt-% up to 50 wt-% of PUFA (and/or salts thereof), based on the total weight of the core.

The shape of the core as well as of the coated particles is also not an essential feature of the present invention. The shape can be sphere-like or any other form (also mixtures of shapes). Usually and preferably the particles are sphere-like.

The coating system according to the present invention is layered around the core. Usually (and ideally) the coating covers the whole surface of the particle. Further-more, the layer is usually (and ideally) equally thick on the surface of the core.

All the preferences for the coating system apply to the above mentioned compositions.

The size of the core as well as the size of the coated particle is not an essential feature of the present invention.

The coated particles are usually of such a size that tablets can be compacted.

A suitable size is between 50-1000μm (preferably 100-800 μm); the size is defined by the diameter of the longest dimension of the particle and measured by commonly known method (like laser diffraction).

Therefore the present invention relates to a coated composition (CC4), which is coated composition (CC), (CC1), (CC1′), (CC2), (CC3) or (CC3′), wherein the average articles size of the coated particles is between 50 and 1000 μm.

Therefore the present invention relates to a coated composition (CC4′), which is coated composition (CC), (CC1), (CC1′), (CC2), (CC3) or (CC3′), wherein the average articles size of the coated particles is between 100 and 800 μm.

All particle sizes are determined by laser diffraction technique using a “Mastersizer 3000” of Malvern Instruments Ltd., UK. Further information on this particle size characterization method can e.g. be found in “Basic principles of particle size analytics”, Dr. Alan Rawle, Malvern Instruments Limited, Enigma Business Part, Grovewood Road, Malvern, Worcestershire, WR14 1XZ, UK and the “Manual of Malvern particle size analyzer”. Particular reference is made to the user manual number MAN 0096, Issue 1.0, November 1994. If nothing else is stated all particle sizes referring are Dv90 values (volume diameter, 90% of the population resides below this point, and 10% resides above this point) determined by laser diffraction. The particle size can be determined in the dry form.

The coated compositions (CC), (CC1), (CC1′), (CC2), (CC3), (CC3′), (CC4) and (CC4′) can be produce according to very well-known processes, such spay drying, drum drying, spray granulation, agglomeration or beadlet. Generally, the coated particles are produced as following:
(i) the core (which comprises PUFA and/or PUFA salts) is produced by commonly known and widely used processes,
(ii) the cores (solid particles) are coated in a second step by a solution, dispersion or slurry of the coating material (protein hydrolysate). The solution, dispersion or slurry is usually made by dissolving or suspending the coating material in water or an aqueous solution. Other suitable solvents like alcohol-water mixture may be used as well. Coating can be applied by well-known technologies like fluid-bed coating or Wurster coating,
(iii) afterwards the coated particles are dried and can be used for further purposes.

The coated compositions (CC), (CC1), (CC1′), (CC2), (CC3), (CC3′), (CC4) and (CC4′) invention can be used in any kind of formulations, wherein the use of such fat soluble ingredients is useful. Usually in food products, feed products, dietary supplements and/or pharmaceutical products.

Therefore the present invention relates to the use of coated compositions (CC), (CC1), (CC1′), (CC2), (CC3), (CC3′), (CC4) and (CC4′) in the production of food products, feed products, dietary supplements and/or pharmaceutical products.

Therefore the present invention relates to the production of food products, feed products, dietary supplements and/or pharmaceutical products, wherein coated compositions (CC), (CC1), (CC1′), (CC2), (CC3), (CC3′), (CC4) and (CC4′) are used.

The food products, feed products, dietary supplements and/or pharmaceutical products can be in any form (liquid, gel-like or solid).

And as described above these coated composition do not “smell” fishy after storage.

The compositions according to the present invention can also be used as or used in dietary supplements. The dietary supplements can be in any form.

The coated compositions according to the present invention can also be used in pharmaceutical products. The pharmaceutical product can be in any galenical form, usually in the form of tablets.

A further embodiment of the present invention relates to food products, feed products, dietary supplements and/or pharmaceutical products, comprising at least one coated composition (CC), (CC1), (CC1′), (CC2), (CC3), (CC3′), (CC4) and/or (CC4′).

The invention is illustrated by the following Example. All temperatures are given in ° C. and all parts and percentages are related to the weight.

### EXAMPLE 1

An aqueous solution containing 5.5 wt-% of whey protein hydrolysate is produced by dissolving/dispersing whey protein hydrolysate in water.

150 g beadlets containing ca 400 mg/g PUFA (mainly DHA and EPA) are fluidized in a lab-scale fluid-bed processor using Wurster technology. The whey protein hydrolysate solution is sprayed on the fluidized particles at a product temperature of 55-64° C. After spraying, the product is dried in the processor at 49-59° C., A free flowing powder of coated beadlets is obtained. The product has a neutral smell, no fishy or rancid smell can be observed.

1. A coating system comprising at least 10 wt-%, based on the total weight of the coating system, of at least one protein hydrolysate.

2. A coated composition comprising
(a) a core, which is a sold formulation comprises at least one PUFA (and/or salts thereof) and
(b) a coating system according to claim 1.

3. The coated composition according to claim 2, comprising
(a) 70 to 99.5 wt-%, based on the total weight of the composition, of core and
(b) 0.5 to 30 wt-%, based on the total weight of the composition, of coating system.

4. Process of production of a coated composition according to claim 2, wherein
(i) the cores (which comprise PUFA (and/or salts thereof) are produced by beadlet process, spray drying or spray-granulation in a first step.
(ii) these cores (solid particles) are coating in a second step by a solution, dispersion or slurry of the coating material (hydrolysate), and
(iii) afterwards the coated particles are dried.

5. Use of the coated particles according to claim 2 in food products, feed products, dietary supplements and/or pharmaceutical products.

6. Food products, feed products, dietary supplements and/or pharmaceutical products, comprising at least one coated composition according to claim 2.
